(19) United States
(12) Patent Application Publication
(10) Pub. No.: US 2014/0028063 A1
(43) Pub. Date: Jan. 30, 2014

(54) HEADREST COVER

(71) Applicant: Paul A. Firestone, Roosevelt Island, NY (US)

(72) Inventor: Paul A. Firestone, Roosevelt Island, NY (US)

(21) Appl. No.: 13/724,067

(22) Filed: Dec. 21, 2012

Related U.S. Application Data
(60) Provisional application No. 61/580,033, filed on Dec. 23, 2011.

(51) Int. Cl. A47C 7/62; A61L 9/12

(52) U.S. Cl. CPC A47C 7/386; A61L 9/12
USPC 297/188.01

(57) ABSTRACT

The headrest cover is used on a headrest of a chair to provide protection between the head of the current user and the previous user of the chair. The headrest cover is a planar, flexible piece of fabric having a pocket which holds tapes, scented blotter paper or scented capsules and absorbent pads. The tape is used to attach the headrest cover to the headrest of a chair. The scented capsules and blotter paper hold a scented liquid or oil. Once blotter paper is removed from its package, or the capsule is broken the liquid scent is poured onto the absorbent pad and the blotter paper or pad is placed back into the pocket of the cover. The scent can than emanate from the pocket and cover foul odors that may find their way to the nose of the user.

### FIELD OF THE INVENTION

This Invention relates to covers for headrests of chairs, especially chairs used in public transport such as airplanes, trains, and long range buses.

### BACKGROUND OF THE INVENTION

Every woman's hair saloon and men's barber shop in the United States is governed by rules of sanitation of that municipality.

According to municipality ordinances, all combs and hairbrushes must be sterilized by hair establishments after being used in grooming the client. Sterilization by ultraviolet rays or sterilizing antiseptic liquid cleanses the grooming instrument, preventing the spread of another client's dirt or oils, disease or contamination to the new client's scalp. Few, if any, public transports provide a sterile cover as a headrest of its passenger seats. Furthermore, especially in the air re-circulation systems of airplanes, individual odors of the passengers tend to diffuse and an adjacent passenger is subject to these diffusing odors.

### SUMMARY OF THE INVENTION

There is a need for a sterile cover for the headrest of chairs which are used repetitively by sequential users. Such chairs are typically found in public transport. Furthermore, there is a need for blocking or masking the individual odor of adjacent passengers where there is a plurality of seats that are adjacent to one another.

To prevent the spread of head lice, scalp infection, dirty hair and pomades that the last person may have deposited on a headrest of a chair of a public transport like a plane, bus, or train, a single personal headrest cover is utilized by each person to allow use of the headrest for comfort with assurance against any personal contaminants present. The present Invention corrects the omission making the travel time experience more pleasant, providing a comfortable and hygienic headrest for sleeping with a variety of aromas that mask the stale, stifling odor of all passengers' flatulence that cannot be eliminated by the normal air systems of the public transport. This is especially true in an airplane's recycling air systems at the altitude that the flight demands.

The headrest cover of the present Invention easily folds into the approximate size of a pocket handkerchief and is the personal property of the individual passenger. Its construction allows it to be secured easily to the headrest by adhesive tape. The headrest cover is washable and therefore usable countless times. A small pocket pouch, secured by a VELCRO fastener, allows for safe storage of the adhesive tape as well as the individual scent carriers which, when opened, allows for a pleasant scent of one's preferred aroma, e.g. spice, evergreen, lavender or citrus, to surround the passenger, freshening the air. The headrest cover of the present Invention provides for comfort and hygienic protection of the passenger.

The headrest cover of the present Invention is intended for use on the headrest of a chair and comprises:
a planar, flexible article having a front side and a back side;
a pocket, attached to the front side of the article;
a plurality of individual strips of adhesive tape in the pocket; and
a plurality of closed scent carriers in the pocket, the scent carriers having a releasable scent.

The planer, flexible article can be natural or man-made, fleece-soft or quilted fabric article.

Preferably, the pocket is sealable with a VELCRO fastener. Preferably, the planar flexible article is padded.

It is preferred that the closed scent carriers are colored coded to correspond to the releasable scent that they contain. For example, the closed scent carrier can be colored green for mint or evergreen, yellow for citrus, pink for apple or spice and purple for lavender.

The scent carrier may be a suitably scented, thick blotter-type paper contained in a plastic-sealed package. The scented blotter paper is impregnated with a scent and sealed in the package to prevent the scent from escaping. The closed scent carrier is opened by removing the scented blotter paper from the packaging. This allows the scent to travel freely in the air. The packaging is a plastic wrap.

Another form of closed scent carriers is plastic capsules held between several layers of a film-diffuser substrate. The plastic capsules, may contain scented oils. The closed scent carrier is opened breaking open the capsule to release the scent onto one substrate and the scent is released into the environment.

The closed scent carrier can also be a capsule having a liquid scent and an absorbent pad. The closed scent carrier is opened by breaking the capsule and pouring the liquid scent from the broken capsule onto the pad. The absorbent pad releases the scent to the surrounding area. The capsule can be a small plastic or foil envelope, such as the type used for individual servings of mustard or ketchup.

The absorbent pad is suitably a cotton pad that is disposable after use. The pocket contains a plurality of these absorbent cotton pads, the number of cotton pads equaling the number of scent capsules in the pocket.

Preferably, the planar flexible article has a design thereon. The article is made of a natural or man-made fabric. One of the unique aspects of the present Invention is that remnants of any soft, washable fabric can be used to manufacture the double-faced headrest cover.

Suitably, strips of tape, preferably double-faced adhesive tape measure approximately one inch in width and three inches in length are used to secure the headrest cover to the chair headrest. The adhesive sides of the tape are covered by a protective cover which is removed when the tape is used to insure the headrest cover adheres to the headrest of the chair.

An alternative design of the headrest cover, replaces the adhesive tapes with two 18-inch, fabric-strip strings which are sewn onto opposite sides of the headrest cover and which are used to tie the headrest cover securely to the back of the head rest of the chair.

An elongated, plastic envelope can be used for packaging and storing the headrest cover and, in such cases, the Invention, carried comfortably in a suit pocket or purse, readily becomes a traveling headrest cover which the traveler than uses on different chairs. The envelope may be similar to a quart-size ZIPLOC bag or a plastic bag measuring 6 inches by 9 inches. The envelope has a safety seal at the point of purchase which is broken open by the buyer.

Preferably, the envelope is transparent so that the design of the headrest cover is visible through the envelope.

The headrest cover has a suitable dimension of 12 inches by 20 inches and can be made from two pieces of fabric that are sewn together. Optionally, there may be a thick internal pad made of a soft fabric filler which is stitched inside so as to provide additional thickness to the headrest cover.

The small pocket of the cover measures approximately four inches by four inches and is preferably sewn to the upper left or upper right-hand corner of the planar flexible article. The pocket is suitably made of a mesh material such that when the open scent carrier is placed into the pocket, the scent can escape from the pocket and mask the surrounding odors. The pocket is used to store the tape strips, and unopened scent carriers.

The cover can suitably be made of recyclable material.

In order to use the cover of the present Invention, the user opens the pocket, removes a closed scented carrier, opens the scent carrier. The open scent carrier is then put back into the pocket. The adhesive tape is used to adhere the cover onto the headrest of a chair. The scent then emanates from the open scent carrier pad through the walls of the pocket so as to diffuse around the passenger sitting in a chair.

Suitably, the scent is an oil based scent which can diffuse up to three hours preventing the passenger from being overwhelmed by the surrounding foul smells that accumulate in the enclosed space.

These and other aspects of the present Invention may be more readily understood by reference to one or more of the following drawings.

### DETAILED DESCRIPTION OF THE DRAWINGS

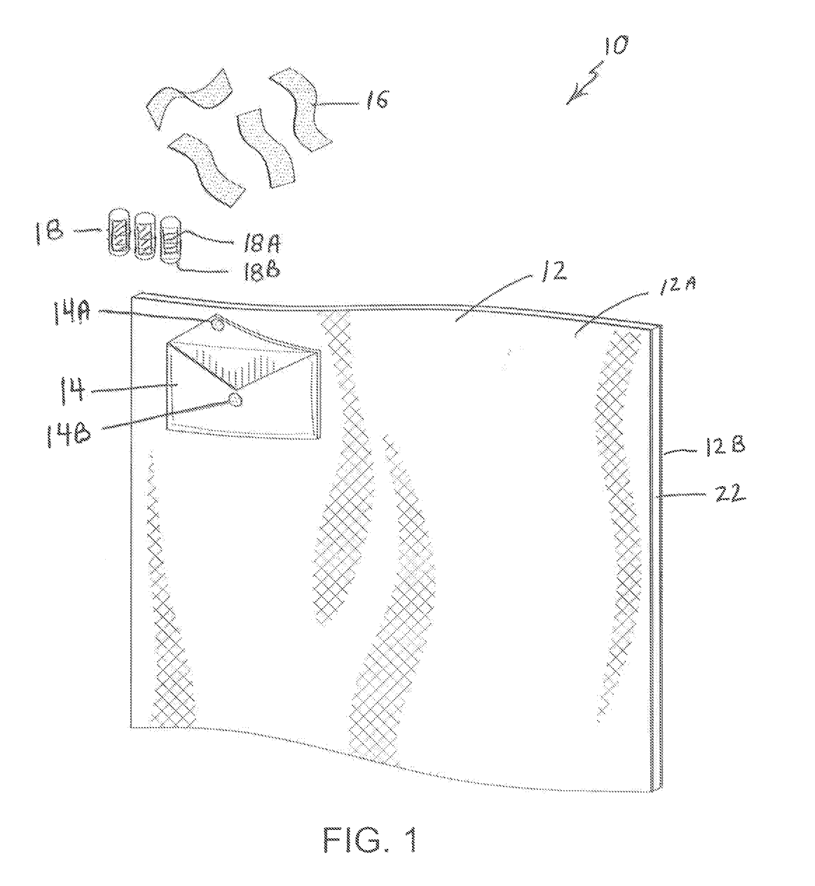

FIG. 1 illustrates the cover with its various components;

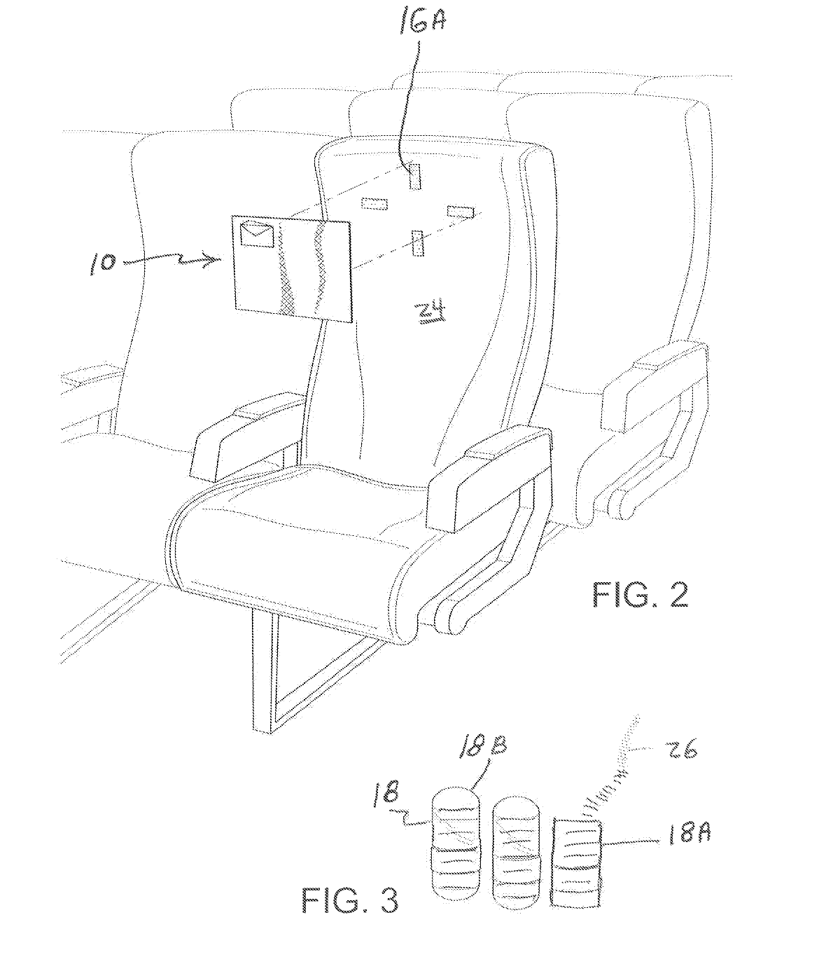

FIG. 2 illustrates the cover being fixed to the headrest of a chair with double sided tape;

FIG. 3 illustrates a broken capsule with scent emanating therefrom;

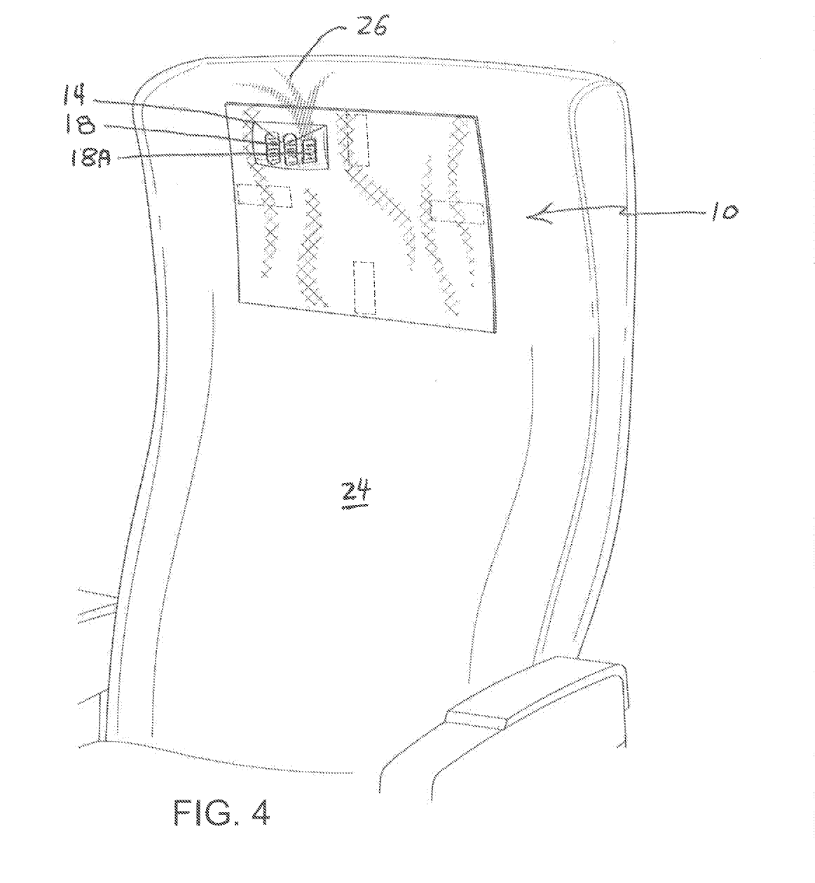

FIG. 4 illustrates the cover fixed to the back of a chair with double sided tape and scent emanating from the pocket of the cover;

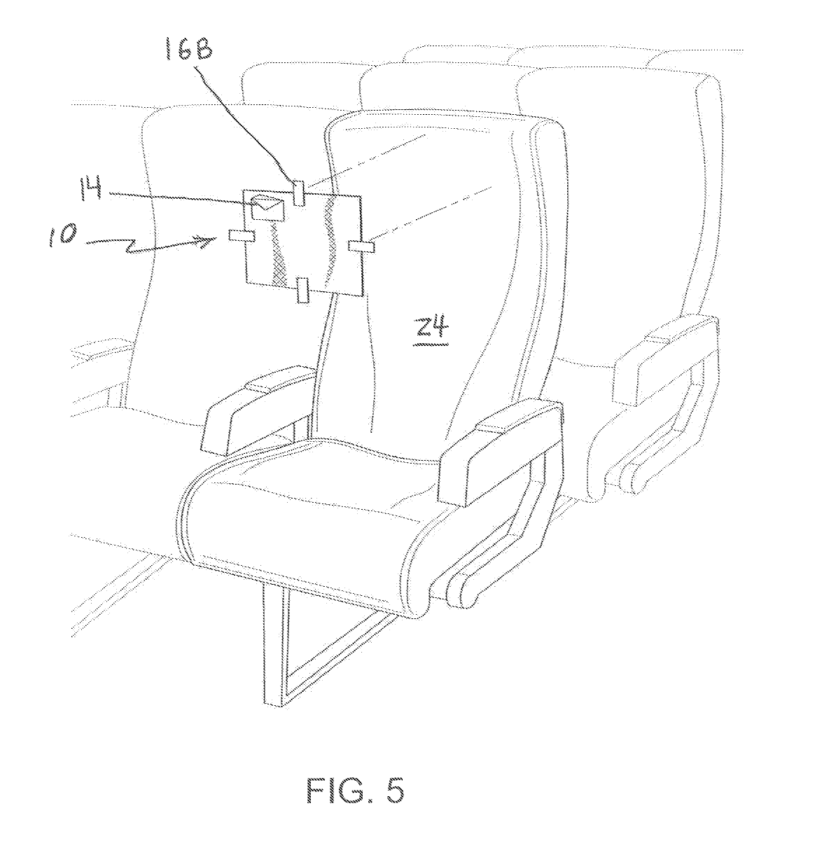

FIG. 5 illustrates the cover being fixed to the headrest of a chair using single sided tape;

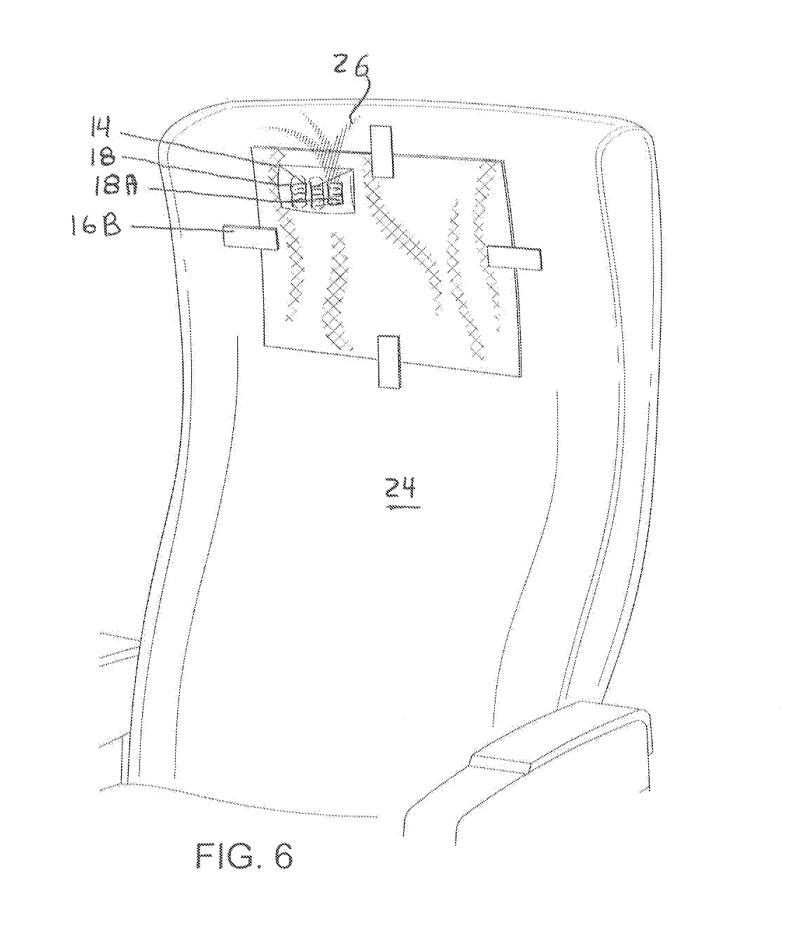

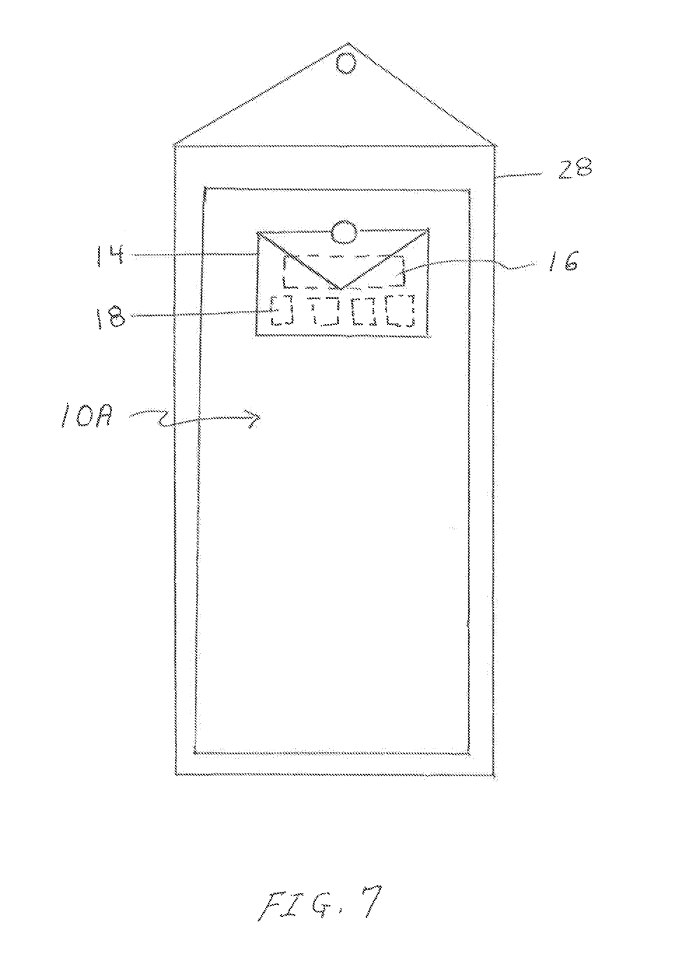

FIG. 6 illustrates a cover fixed to the headrest of a chair with single sided tape and scent emanating from the pocket;

FIG. 7 illustrates the cover folded inside an envelope for purposes of carrying;

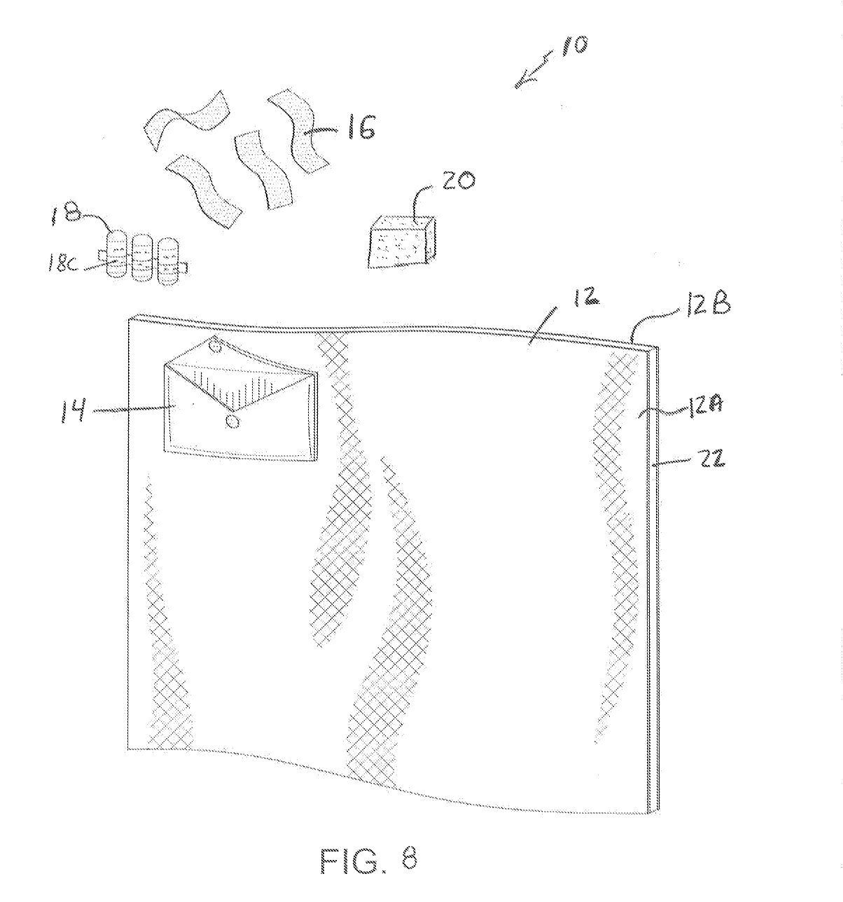

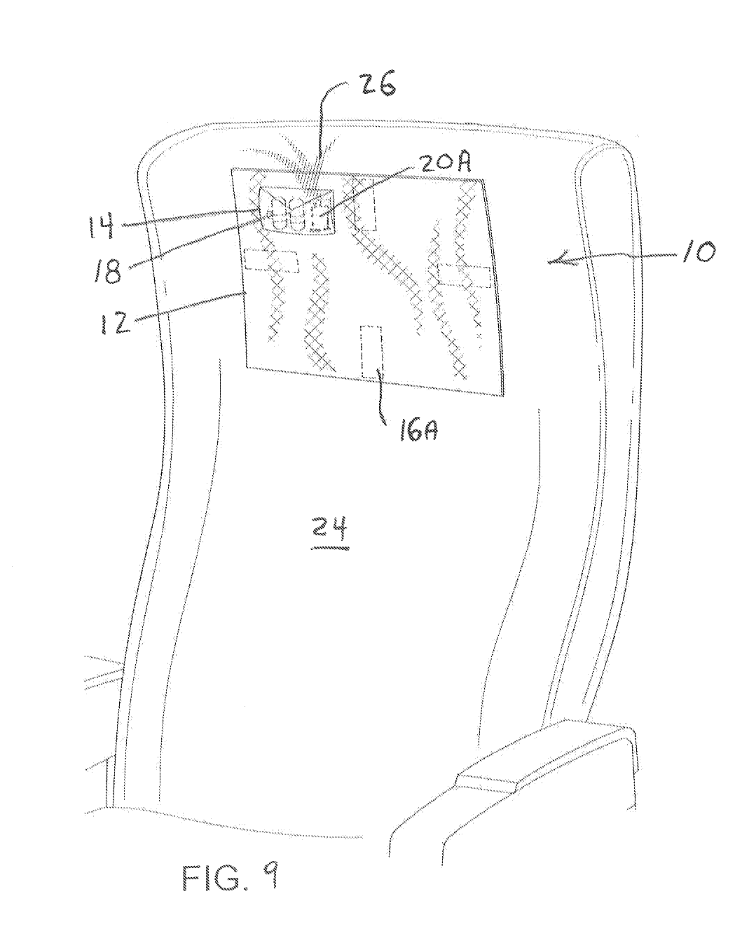

FIG. 8 illustrates the cover with capsules and an absorbent pad as the scent carriers;

FIG. 9 illustrates the cover of FIG. 8 attached to a chair;

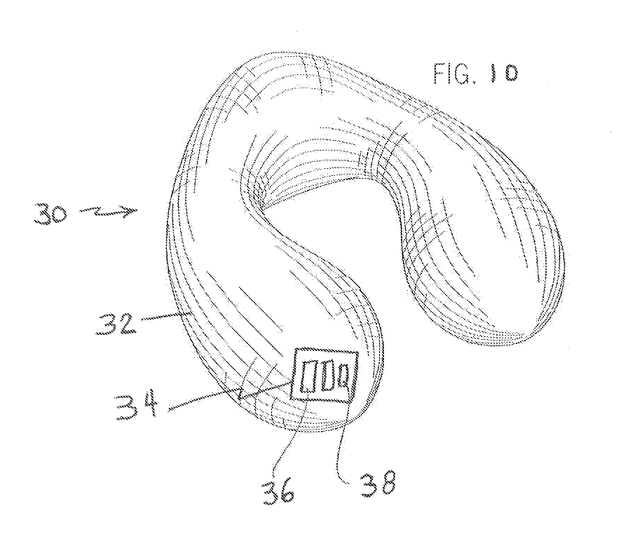

FIG. 10 illustrates a neck pillow in accordance with the invention; and

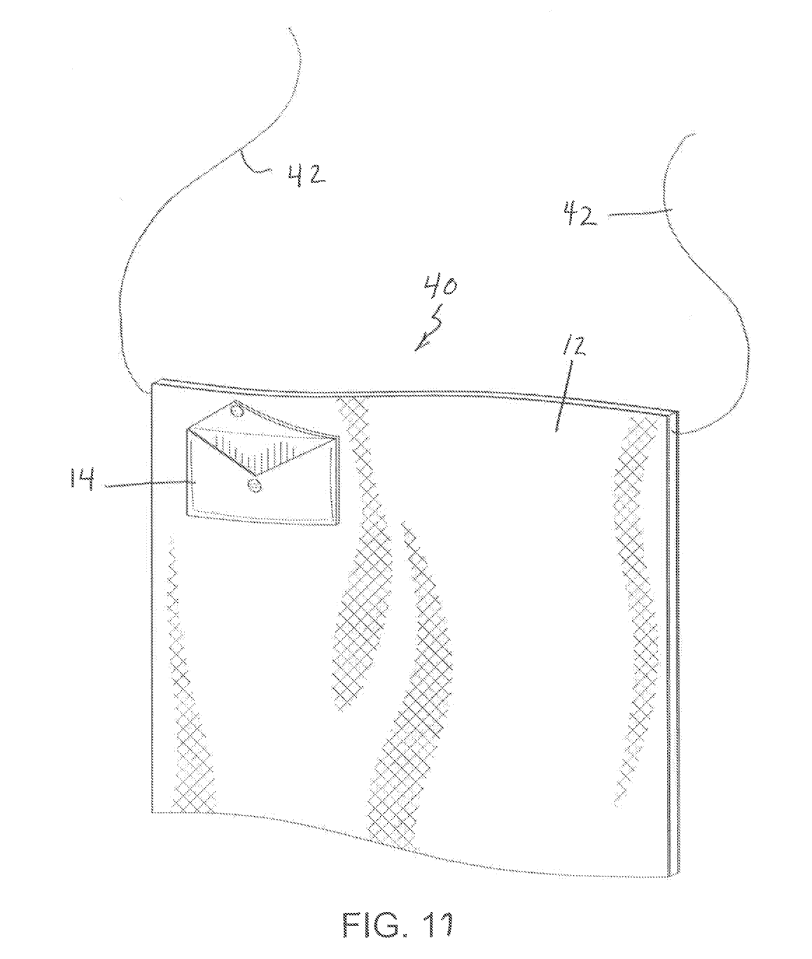

FIG. 11 illustrates the cover with string to attach to chair.

### DETAILED DESCRIPTION OF THE INVENTION

FIG. 1 illustrates headrest cover 10 which comprises planar flexible article 12 having mesh pocket 14 sewn thereon. Pocket 14 is intended to be large enough to carry adhesive tape strips 16 plurality of closed scent carriers 18. Closed scent carrier 18 has scented blotter paper 18A and packaging 18B which seals the scent carrier.

Article 12 has a front side 12A and a backside 12B with padding 22 sewn between front side 12A and backside 12B.

Suitably, padding 22 is a soft, cotton-type padding which provides some additional cushioning to thr headrest cover 10. Optionally, article 12 has no padding and two pieces of fabric remnant are used to make up front side 12A and backside 12B. These pieces of remnant are sewn together to form article 12.

One of the unique aspects of the present Invention is that remnants of fabric are used to make the headrest cover 10. By using remnants of fabric, headrest cover 10 can have a plurality of designs which can be a unique selling feature of the Invention. In fact, it is contemplated that the headrest cover 10 can be sold as souvenirs, or gifted as corporate promotional give-aways, or merchandised as gifts based on holidays or the like. For example, during the Fourth of July, headrest cover 12 may bear a patriotic symbol; during Easter, bunnies or colored eggs; and during Thanksgiving, a turkey.

Pocket 14 is attached to front 12A of article 12. Preferably, pocket 14 is attached to the upper left or upper right-hand corner of article 12. Pocket 14 is large enough to carry a plurality of strips of tapes 16, a plurality of scent carriers 18. Pocket 14 is closed using a VELCRO tabs 14A and 14B.

Tape 16 can be either single sided adhesive tape or double sided adhesive tape. The adhesive tape has covers on the adhesive side of the tape which are removable upon use of the tape.

Scent carriers 18 can be the same or mixed with respect to the scent that they carry. A scented liquid is suitably stored in each of the carriers and upon opening of the scent carrier, scent can escape.

Turning to FIG. 2, the headrest cover 10 is shown as being mounted to the headrest of chair 24 by double sided tape 16A. Chair 24 is shown in conventional airplane type setting where there is a plurality of chairs intended to hold a plurality of passengers.

FIG. 3 illustrates open scent carrier, exposed blotter paper 18A with scent 26 emanating therefrom.

FIG. 4 illustrates the headrest cover 10 fixed to the headrest of chair 24. Inside pocket 14 are the remaining unopened scent carriers 18 and opened scent carrier 18A. Scent 26 illustrated emanating through pocket 14 and around the headrest of seat 24.

FIG. 5 illustrates the headrest cover 10 being applied to the headrest of chair 24 by single sided tape 16B.

FIG. 6 illustrates the headrest cover 10 fixed to the headrest of chair 24 by single sided tape 16B and scent 26 emanating from open scent carrier 18A.

FIG. 7 illustrates the traveling arrangement for the headrest cover 10. In FIG. 7, folded headrest cover 10A is inside envelope 28. Inside pocket 14 of folded headrest cover 10A are a plurality of adhesive strips 16, and a plurality of closed scent carrier 18.

Envelope 28 is suitably transparent so that the design on headrest cover 10 is visible through the envelope.

FIGS. 8 and 9, illustrate the embodiment where the scent carrier 18 is a capsule containing a liquid scent 18C and an absorbent pad 20 is used to absorb and deliver the scent. FIG. 9 shows headrest cover 10 fixed to a headrest of seat 24 with pad 20A onto which liquid scent 18C has been poured.

FIG. 10 illustrates a conventional u-shaped neck rest pillow 30 having a removable, washable cover 32 with pocket 34 for holding unopened scent carriers 36 and opened scent carriers 38 for use in accordance with the present invention.

FIG. 11 illustrates where headrest cover 40 has fabric strings 42 to attach headrest cover 10 to chair 24.

The foregoing description of the preferred embodiments of the Invention have been presented for purposes of illustration and description and are not intended to be exhaustive or to limit the Invention to the precise form disclosed. The description has been selected to best explain the principles of the Invention and their practical applications to enable others of skill in the art to best utilize the Invention in various embodiments and various modifications as are suited to the practical particular use contemplated. It is intended that the scope of the Invention not be limited to the Specification but be defined by the claims as set forth below.

I claim:

1. A headrest cover for a headrest of a chair, comprising:
a planar, flexible article having a front side and a backside;
a pocket attached to the front side of the article;
a plurality of individual strips of adhesive tapes in the pocket; and
a plurality of scent carriers in the pocket, the scent carries having a releasable scent.

2. The cover of claim 1, wherein
the article has padding between the front side and the back side.

3. The cover of claim 1, wherein
the front side and the back side of the article has a design thereon.

4. The cover of claim 1, wherein
the front side and the back side of the article are made of fabric remnants which are sewn together.

5. The cover of claim 1, wherein
the scent carriers are color coded to correspond to the releasable scent.

6. The cover of claim 1, wherein
the scent carrier is a blotter-type paper impregnated with a scent.

7. The cover of claim 1, wherein
the scent carrier is a capsule with liquid scent and an absorbent pad.

8. The cover of claim 1, wherein
the planar flexible article is rectangular in shape.

9. The cover of claim 1, wherein
the adhesive tape has adhesives on both sides.

10. The cover of claim 1, wherein
the adhesive tape has adhesive on one side.

11. A traveling headrest cover comprising:
an elongated envelope; and
a folding cover of claim 1 inside the envelope.

12. The traveling headrest cover of claim 11, wherein
the envelope is made of a transparent material;
the cover has a design thereon; and
the design is visible through the envelope.

13. A chair having a headrest comprising:
the headrest cover of claim 1 attached to the headrest by means of the adhesive tape and scent emanating from the pocket in which scented liquid from a broken capsule has been poured onto the absorbent pad.
